(19) United States
(12) Patent Application Publication
(10) Pub. No.: US 2005/0256152 A1
(43) Pub. Date: Nov. 17, 2005

(54) Salt of a sulfonic acid containing clopidogrel and use thereof for the production of pharmaceutical formulations

(76) Inventors: Karlheinz Doser, Buchholz i.d. Nordheide (DE); Klaus Glaenzer, Hamburg (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/510,578

(22) PCT Filed: Feb. 13, 2004
(86) PCT No.: PCT/EP04/01370

§ 371 Date: May 10, 2005

(30) Foreign Application Priority Data
Feb. 13, 2003 (DE) 10305984.9

(51) Int. Cl. C07D 498/02; A61K 31/4743

(52) U.S. Cl. 514/301; 546/114

(57) ABSTRACT

The present invention relates to the salt of a sulfonic acid with clopidogrel and pharmaceutical formulations comprising this salt.

The present invention relates to the salt of a sulfonic acid with clopidogrel, a method for preparing the same and the use thereof for preparing pharmaceutical formulations. The present invention further comprises active ingredient particles with clopidogrel or a pharmaceutically acceptable salt thereof.

Clopidogrel (5-methyl-α-(4,5,6,7-tetrahydro [2,3-c]thienopyridyl)(2-chlorophenyl)acetate) is known from EP-A-0 099 802 as an active ingredient. Clopidogrel acts as a platelet aggregation inhibitor and may therefore be used for the prevention of thromboembolic events such as a stroke or a myocardial infarction.

EP-A-0 281 459 proposes to use inorganic salts of the (S)-(+) clopidogrel, particularly (S)-(+) clopidogrel hydrogen sulfate in pharmaceutical formulations. This document also discloses organic salts of clopidogrel, but these are described as amorphous and/or hygroscopic and difficult to purify.

The (S)-(+) clopidogrel hydrogen sulfate used in pharmaceutical formulations has the disadvantage that concentrated sulfuric acid is required for preparation thereof and that the resulting products react in a superacidic manner because of the acidic proton. These acidic characteristics affect the compatibility with many pharmaceutical adjuvants and thus the stability of drug forms resulting therefrom. Therefore, there is a need for stable forms of clopidogrel which are easy to purify and may be processed readily with different pharmaceutical adjuvants such as drug carriers and additives.

Therefore, it is one object of the present invention to provide clopidogrel in a form which is easy to purify and stable and may be processed readily even at an industrial scale. In addition, interaction with common drug carriers, additives and processing aids should be avoided where possible.

Contrary to the disclosure of EP-A-0 281 459, it has now been found surprisingly that the salt of a sulfonic acid with clopidogrel is suitable under certain conditions for preparing pharmaceutical formulations.

The present invention therefore relates to the salt of a sulfonic acid with clopidogrel at least part of which is present in crystalline form. The present invention further relates to the salt of a sulfonic acid with clopidogrel which is preparable by precipitating the salt from a clopidogrel solution, the solvent comprising a hydrocarbon and/or an ether.

According to the invention, a racemic mixture of the two clopidogrel isomers may be used as the clopidogrel. Alternatively, it is possible to use the pure isomers, the (S)-(+) clopidogrel isomer being preferred.

According to the invention, it has now been found surprisingly that, contrary to the teaching of EP-A-0 281 459, it is possible to incorporate the salt of a sulfonic acid with clopidogrel into pharmaceutical formulations and especially into pharmaceutical formulations for oral administration. Therefore, the invention also comprises using the salt of a sulfonic acid with clopidogrel for preparing a pharmaceutical formulation and pharmaceutical formulations containing such a salt.

The salt of the invention is crystalline at least in part and preferably completely crystalline. In this form, the salt may be purified more easily than in the amorphous form disclosed in EP-A-0 281 459. In addition, it is easier to process crystalline salt into pharmaceutical formulations.

According to the invention, it has also been found that the desired and especially the crystalline salts of a sulfonic acid with clopidogrel may be prepared easily and in a form advantageous for further processing into a pharmaceutical formulation by precipitating the salt from a solution of clopidogrel if the solvent comprises a hydrocarbon and/or an ether. Preferably, the solvent comprises toluene, dioxane, methyl-tert-butyl ether (MTB ether) and/or diethyl ether. It is especially preferred to use mixtures of toluene and acetone, dioxane and ethyl acetate or MTB ether, ethyl acrylate and isopropanol.

For example, the clopidogrel base may be dissolved in toluene and the desired salt precipitated by adding a sulfonic acid solution, for example a benzene sulfonic acid solution in acetone. In another embodiment, both the clopidogrel base and the sulfonic acid, for example benzene sulfonic acid, may be dissolved in dioxane, mixed and the desired salt precipitated by adding ethyl acetate. In yet another embodiment, both the clopidogrel base and the sulfonic acid, for example toluene sulfonic acid, may be dissolved in ethyl acetate, mixed and the desired salt precipitated by adding MTB ether and isopropanol.

According to the method described above, the salt of a sulfonic acid with clopidogrel may be obtained in good yield and purity so that this salt is particularly well suited for preparing pharmaceutical formulations, especially when it is present in crystalline form.

Methane sulfonic acid, ethane sulfonic acid, benzene sulfonic acid, toluene sulfonic acid, such as toluene sulfonic acid and naphthalene sulfonic acid, e.g. α-naphthalene sulfonic acid, are examples of the sulfonic acids used for the salts of the invention. Benzene sulfonic acid and toluene sulfonic acid are preferred.

It has also been found that the salt of a sulfonic acid with clopidogrel has particularly advantageous properties with regard to crystallinity if it contains solvent molecules. The solvent molecules intercalated in solvate form in the salt originate from the solution from which the salt was precipitated. Preferably, the salt contains toluene or dioxane.

The salt of the benzene sulfonic acid with clopidogrel precipitated from toluene contains toluene molecules.

The 10 most intensive peaks of the X-ray powder spectrum of this salt have the following 2θ values:

| Relative intensity | 2θ |
|---|---|
| 99.11 | 10.80 |
| 100.00 | 12.08 |
| 96.77 | 16.09 |
| 62.57 | 16.66 |
| 84.58 | 20.22 |
| 93.53 | 21.50 |
| 66.00 | 22.56 |
| 78.33 | 22.91 |
| 81.82 | 23.45 |
| 56.15 | 24.92 |

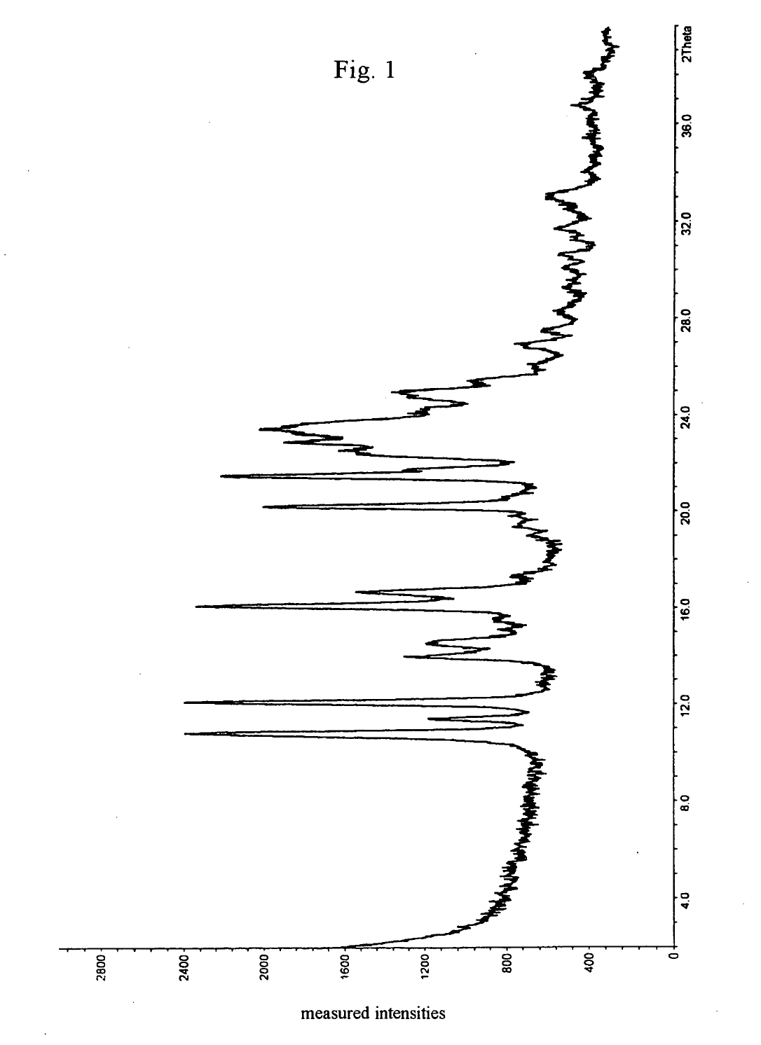

The X-ray powder spectrum which was obtained with a STEO STADI P transmission diffractometer using copper Kα radiation is shown in the attached FIG. 1.

The benzene sulfonic acid salt precipitated from dioxane contains dioxane molecules. The 10 most intensive peaks of the X-ray powder spectrum of this salt have the following 2θ values:

| Relative intensity | 2θ |
|---|---|
| 51.66 | 10.78 |
| 54.15 | 10.87 |
| 90.13 | 12.13 |
| 50.83 | 14.34 |
| 50.27 | 16.43 |
| 76.03 | 21.57 |
| 81.19 | 22.87 |
| 100.00 | 23.06 |
| 54.18 | 23.72 |
| 54.05 | 25.17 |

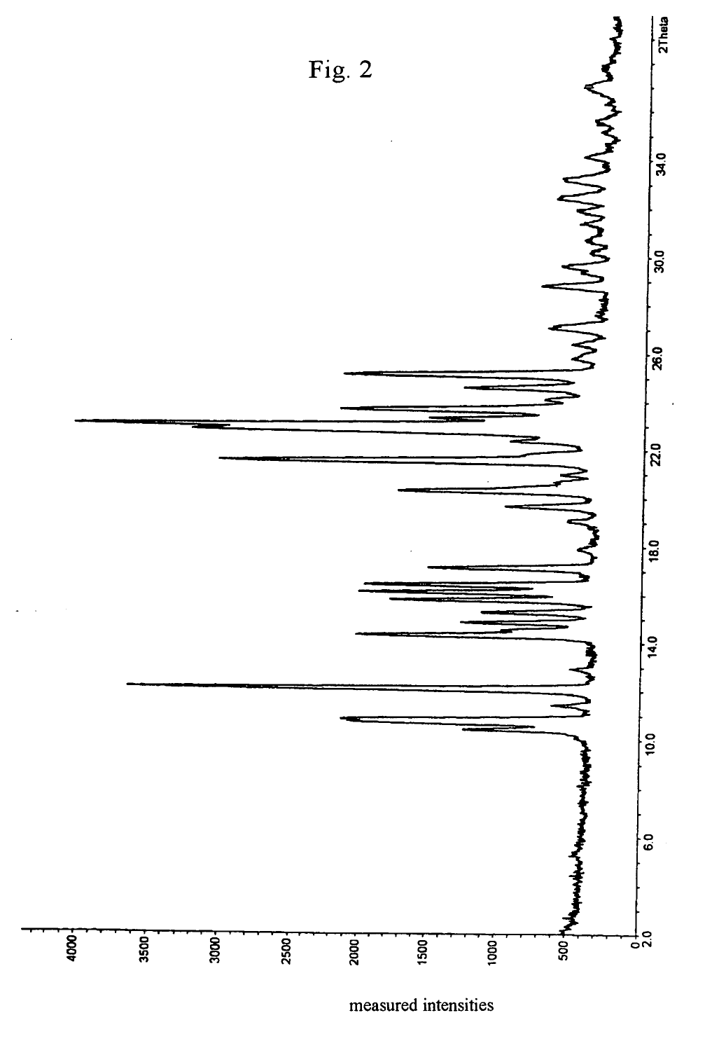

The X-ray powder spectrum of this salt measured as described above is shown in the attached FIG. 2.

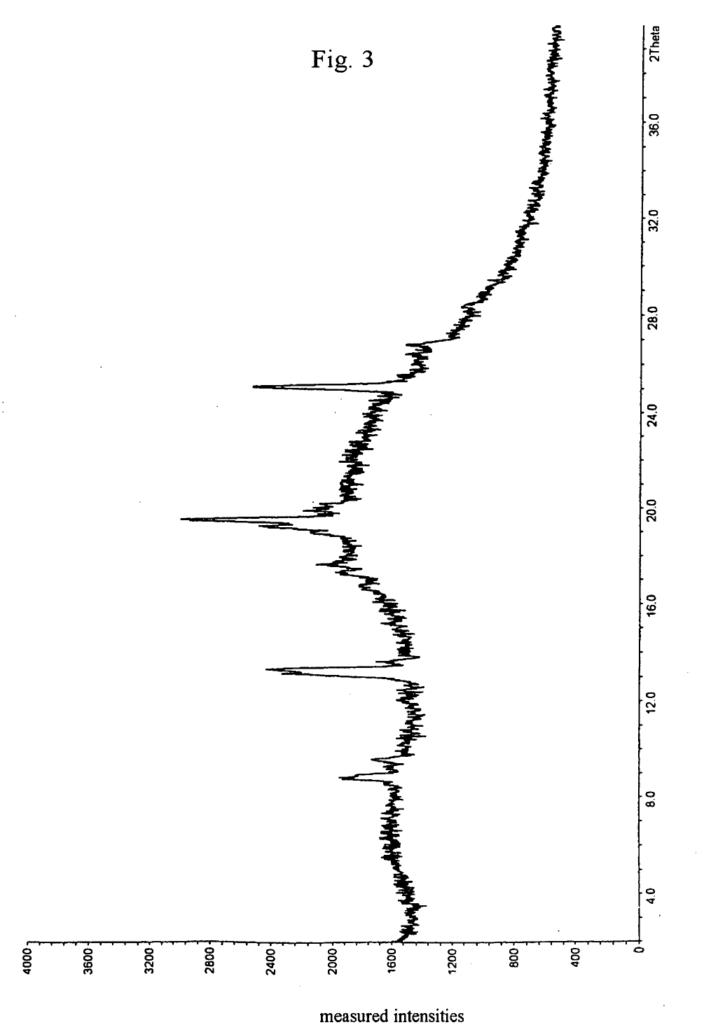

The partially crystalline salt of the toluene sulfonic acid with clopidogrel shows the X-ray powder spectrum measured as above as shown in the attached FIG. 3. The 10 most intensive peaks of the X-ray powder spectrum of this salt have the following 2θ values:

| Relative intensity | 2θ |
|---|---|
| 80.54 | 13.13 |
| 83.15 | 13.28 |
| 67.75 | 17.28 |
| 70.05 | 17.64 |
| 73.78 | 18.96 |
| 84.65 | 19.21 |
| 100.00 | 19.48 |
| 75.95 | 19.87 |
| 71.09 | 20.12 |
| 86.48 | 25.06 |

In addition, it was found that the salt of a sulfonic acid with clopidogrel is obtained in particularly high purity when compared with other clopidogrel salts. A besylate salt crystallised from dioxane, for example, will contain only 0.085% of impurities (according to HPLC). Therefore, the salt of the invention is well suited for preparing pure clopidogrel. The invention thus also relates to a method for purifying clopidogrel wherein contaminated clopidogrel or a salt thereof, optionally after release of the clopidogrel base, is converted into the salt of a sulfonic acid with clopidogrel and, if desired, the clopidogrel base is then released from the isolated salt of the sulfonic acid and/or converted into another salt. It is preferred to use the besylate salt.

It is a further aspect of the invention to provide clopidogrel or a pharmaceutically acceptable salt thereof in a form which is easy to process further. In the invention, this is achieved by applying the salt onto a solid adsorbent. As a result, active ingredient particles are obtained which are easy to pour and dose.

A suitable adsorbent is any physiologically and pharmaceutically acceptable, preferably particulate solid capable of adsorbing clopidogrel or a salt thereof. Preferably, the solid is a free-flowing powder which may be processed easily into oral pharmaceutical formulations.

Examples of physiologically and pharmaceutically acceptable solids are, for example:
1. Natural or processed adsorbents from the group of clays (clay materials) and other earths and minerals, e.g. attapulgites, aluminium-magnesium silicates (Carrisorb®, Gelsorb®), magnesium-aluminium silicates (Pharmasorb®Veegum®), magnesium silicates (talcum), calcium silicates, bentonites, kaolin, magnesium trisilicates, montmorillonites, china clays (bolus), sepiolites (meerschaum)
2. Silica gels, kieselguhr, silicic acids
3. Colloidal (highly disperse) silicic acids (hydrophobic or hydrophilic Aerosile®, Cab-o-sile®)
4. Celluloses, modified celluloses, finely and micro-crystalline celluloses and cellulose derivatives, cellulose acetate, cellulose fatty acid esters, cellulose nitrates, cellulose ethers (carboxymethyl celluloses, ethyl celluloses, hydroxyethyl celluloses, hydroxypropyl celluloses, methyl celluloses, methylethyl celluloses, methylhydroxypropyl celluloses)
5. Sugars and sugar derivatives (mono- and polysaccharides), lactoses, dextranes, dextrose, cyclodextrines
6. Native maize, rice, tapioca, wheat and potato starches and derivatives thereof, dextrines, pre-gelatinised, fully or partially hydrolysed starches
7. Solid polyols, especially mannitol or sorbitol
8. Polyacrylates, acrylic acid polymers or copolymers
9. Phosphates, sulfates, carbonates, gluconates, oxides of alkaline or alkaline earth metals as well as physiologically acceptable heavy and transition metals
10. Guar flour, guar gum
11. Locust bean flour (carob flour, carob gum)
12. Alginic acid, alginates and seaweed flour
13. Tragacanth
14. Carbo vegetabilis (coal)
15. Pectines and amylopectines
16. N-Vinylpyrrolidone polymers such as povidone or crospovidone.

The adsorbents may be used singly or in blends of two or more adsorbents. Besides the adsorbent, the active ingredient particles of the invention may also comprise the usual pharmaceutical adjuvants, for example for the preparation of direct compression mixtures or for the preparation of granulates for further processing into drugs. Alternatively, the active ingredient particles may be mixed with suitable adjuvants after preparation and then processed into pharmaceutical formulations.

Especially preferred adsorbents are certain lactoses (e.g. Lactopress®), certain mannitols (e.g. Mannogem®) and certain celluloses (e.g. Celphere®), particularly Lactopress®. A granulate on the basis of silica prepared by the pyrogenic route, even though possible, is preferably not used as the carrier medium.

Suitable humectants may be used to control desorption. In order to improve stability, it is possible, for example, to add antioxidants such as ascorbic acid and salts thereof. Other suitable adjuvants are emulsifiers, solvents and solubilisers.

The active ingredient particles may, for example, be recovered from a solvent wherein the adsorbent is insoluble or poorly soluble and the clopidogrel or the salt thereof is soluble. For this purpose, the adsorbent may be suspended in the solvent. The clopidogrel or the salt thereof may be dissolved directly in the solvent either before or after the suspending step. The active ingredient may be added either directly or as a solution in the same or a different solvent. After that, the active ingredient particles comprising the clopidogrel or the salt thereof applied on the adsorbent are recovered from the solvent, for example by evaporating the solvent.

Suitable solvents are all customary solvents wherein the selected adsorbent is insoluble or poorly soluble and the clopidogrel or the salt thereof is soluble. For example, the solvents described above in connection with the preparation of the salt may be used.

In an alternative embodiment of the method of the invention for preparing active ingredient particles, the last stage of the synthesis of clopidogrel is carried out in the presence of the adsorbent. This makes it possible to prepare the desired active ingredient particles without an isolating intermediate step. It is also possible, for example, to mix clopidogrel and an acid with the suspension of the adsorbent. In this process, the clopidogrel and the acid may each be dissolved separately in a solvent and added to the suspension either simultaneously or one after the other. Alternatively, the clopidogrel and the acid may be added to the suspension in pure form. It is also possible to premix individual components and to then add them to the suspension in joint form.

The weight ratio between the adsorbent and the clopidogrel or the salt thereof adsorbed thereupon is not essential for the invention and may be selected by the skilled practitioner depending on the desired use. If it is intended to process the mixture into oral pharmaceutical formulations, care should be taken that sufficient clopidogrel is coated on the adsorbent so that the desired dose in the unit dosage form may be obtained. For example, the weight ratio of clopidogrel or the salt of clopidogrel based on the free clopidogrel base to the adsorbent may be in the range from 2:1 to 1:6 (i.e., for example, 1 part by wt. of clopidogrel base per 6 parts by wt. of adsorbent), preferably in the range from 1:1 to 1:3.

Preferred salts of the clopidogrel are hydrogen sulfate, hydrochloride, mesylate, besylate, tosylate and napsylate.

The present invention is illustrated, but not limited by the following examples.

The X-ray powder spectra in the examples were obtained by means of a STOE STADI P transmission diffractometer with copper Kα radiation; the NMR data were obtained with the aid of a Varian Unityplus 300 device and the CHN data by means of a Carlo Erba Analyzer 1106.

### EXAMPLE 1

Preparation of Clopidogrel Benzene Sulfonate from Acetone/Toluene

4.0 g (12.5 mmol) of clopidogrel base were dissolved in 30 ml of toluene. Then 2.0 g (12.5 mmol) of anhydrous benzene sulfonic acid in 10 ml of acetone were added. After some time and grinding with a glass rod, the product solidifies and may be drawn off by suction. The product was dried over night under vacuum at the pump system in the desiccator.

Yield: 67% Melting point 87 to 90° C.

NMR (ppm) 2.35 (toluene) 3.0-3.5 and 3.8-4.3 (4H), 3.79 (3H), 4.8-5.2 (1H), 5.69 (1H), 6.6-6.8 (1H), 7.2-8.0 (12H).

The X-ray powder spectrum of this salt is shown in FIG. 1.

Upon further drying until all of the toluene has been removed from the salt, the crystal structure collapses and amorphous clopidogrel benzene sulfonate is obtained.

### EXAMPLE 2

Preparation of Clopidogrel Benzene Sulfonate from Dioxane

To 109.2 g (339.7 mmol) of clopidogrel base dissolved in dioxane, a solution of 53.7 g (339.7 mmol) of anhydrous benzene sulfonic acid in 100 ml of dioxane is added with stirring at 10° C. 250 ml of ethyl acetate are added to this solution and the solution placed into a deep-freezer over night. The solution is allowed to warm to room temperature and the crystallisate removed by suction, followed by washing with ethyl acetate. The product is dried under vacuum at room temperature for 48 hours.

Yield: 71% Melting point 93 to 95° C.

Elementary analysis

| | | |
|---|---|---|
| | Calculated for clopidogrel | |
| Values (%) | besylate * ½ dioxane | Found |

| | | | |
|---|---|---|---|
| C | 55.01 | 55.28 | 55.03 |
| H | 5.00 | 5.12 | 4.99 |
| N | 2.67 | 2.62 | 2.53 |

NMR (ppm) 3.0-3.5 and 3.8-4.3 (4H), 3.79 (3H), 4.8-5.2 (1H), 5.68-5.72 (1H), 6.6-6.8 (1H), 7.2-8.0 (12H), 3.70 (4H; ½ dioxane)

The X-ray powder spectrum of this salt is shown in FIG. 2.

### EXAMPLE 3

Preparation of Clopidogrel Toluene Sulfonate from MTB Ether

4.0 g (12.5 mmol) of clopidogrel base are dissolved in 50 ml of ethyl acetate. Then a solution of 2.2 g (12.5 mmol) of toluene sulfonic acid (anhydrous) in 30 ml of ethyl acetate is added. About 50 ml of ethyl acetate are distilled off under vacuum and 150 ml of MTB ether and 5 ml of isopropanol are added and the residue is stirred until a solid mass is obtained. Removal by suction is followed by drying under vacuum at room temperature.

Yield: 62% Melting point 78 to 82° C.

The X-ray powder spectrum of this salt is shown in FIG. 3.

### EXAMPLE 4

Stability Tests

4.1 The stress stability of various salts of the clopidogrel was tested under different conditions. The salts used were the form II of clopidogrel hydrogen sulfate (known as the most stable so far), clopidogrel hydrochloride (prepared according to EP 0 281 459), amorphous clopidogrel benzene sulfonate and crystalline clopidogrel benzene sulfonate (as prepared in the above example 2). The following tests were conducted:

Stability Under Acidic Conditions

50 mg of each salt were weighed into a volumetric flask (100 ml) and 2 ml of 1N HCl were added. Then the flask is kept either at room temperature for 5 hours or at 80° C. for 5 hours. After the end of each experiment and, optionally, cooling to room temperature, 2 ml of 1N NaOH are added and mobile phase is added up to 100 ml.

The result is determined by means of HPLC.

Stability Under Basic Conditions

50 mg of the salt concerned are weighed into a volumetric flask (100 ml) and 2 ml of 1N NaOH are added. Then the flask is held either at room temperature for 5 hours or at 80° C. for 5 hours. After the end of each experiment and, optionally, cooling to room temperature, 2 ml of 1N HCl are added and mobile phase is added up to 100 ml.

The result is determined by means of HPLC.

Stability Under Oxydative Conditions

50 mg of the salt concerned are weighed into a volumetric flask (100 ml) and 2 ml of 3% H₂O₂ added. Then the flask is kept either at room temperature for 5 hours or at 80° C. for 5 hours. After the end of each experiment and, optionally, cooling to room temperature, the mobile phase is added up to 100 ml.

The result is determined by means of HPLC.

Stability Under Neutral Conditions

50 mg of the salt concerned are weighed into a volumetric flask (100 ml) and 2 ml of water added. Then the flask is kept either at room temperature for 5 hours or at 80° C. for 5 hours. After the end of each experiment and, optionally, cooling to room temperature, the mobile phase is added up to 100 ml.

The result is determined by means of HPLC.

Stability Under the Influence of Heat

50 mg of the salt concerned are weighed into a volumetric flask (100 ml) and held at 80° C. for 20 hours. After the end of each experiment and cooling to room temperature, the mobile phase is added up to 100 ml.

The result is determined by means of HPLC.

In all cases, the HPLC measurements were carried out under the following conditions with UV detection:

| | | |
|---|---|---|
| Column: | Hypersil BDS 5 μm, 250 · 4.6 mm | |
| Mobile phase: | Methanol | 650 ml |
| | 0.05 M 1-octane sulfonic acid-Na salt | 350 ml |
| | (adjusted to a pH of 2.5 with | |
| | triethyl amine and phosphoric acid) | |
| Flow rate: | 1 ml/min | |
| Temperature | Room temperature | |
| of the column: | | |
| Wavelength: | 215 nm | |
| Injection volume: | 20 μl | |
| Retention time: | approx. 15 min. | |

The results of these tests are summarised in the following tables 1 to 4.

Clopidogrel Hydrogen Sulfate

TABLE 1

| Condition | Room temperature | 80° C. |
|---|---|---|
| acidic | 0.32% | 2.96% |
| alkaline | 0.32% | 59.48% |
| oxidising | 0.33% | 3.50% |
| neutral | 0.40% | 1.63% |
| heat | — | 0.31% |

Clopidogrel Hydrochloride

TABLE 2

| Condition | Room temperature | 80° C. |
|---|---|---|
| acidic | 1.86% | 3.31% |
| alkaline | 1.86% | 72.89% |
| oxidising | 1.83% | 4.16% |
| neutral | 1.84% | 4.33% |
| heat | — | 32.43% |

Clopidogrel Benzene Sulfonate (Amorphous)

TABLE 3

| Condition | Room temperature | 80° C. |
|---|---|---|
| acidic | 0.64% | 2.36% |
| alkaline | 0.64% | 25.04% |
| oxidising | 0.83% | 2.94% |
| neutral | 0.85% | 3.01% |
| heat | — | 11.52% |

Clopidogrel Benzene Sulfonate (Crystalline)

TABLE 4

| Condition | Room temperature | 80° C. |
|---|---|---|
| acidic | 0.14% | 2.76% |
| alkaline | 0.14% | 28.05% |
| oxidising | 0.13% | 3.98% |
| neutral | 0.19% | 4.18% |
| heat | — | 4.52% |

It is evident that, contrary to the teaching of EP 0 281 459, the amorphous clopidogrel benzene sulfonate has a comparable and, under alkaline conditions, even a considerably increased stability in comparison with the hydrogen sulfate and hydrochloride salts of clopidogrel. In addition, the stability of the crystalline form of the clopidogrel benzene sulfonate is further increased vis-à-vis that of the amorphous form of this salt, especially at room temperature which is important for storing pharmaceutical products. Crystalline clopidogrel benzene sulfonate is even more stable than clopidogrel hydrogen sulfate, so far known as the most stable one and used in pharmaceutical formulations.

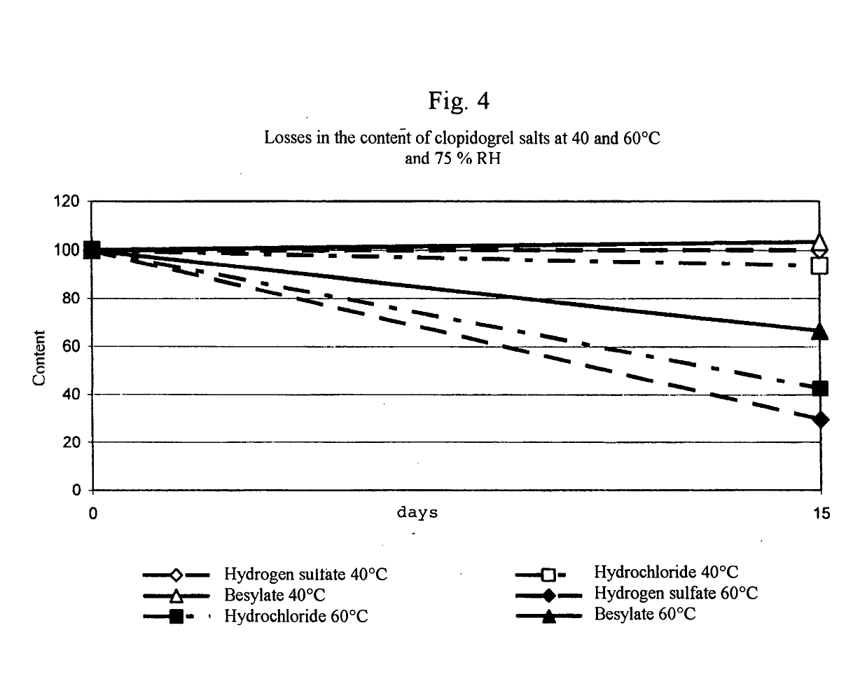

4.2 In addition, the decrease of the clopidogrel hydrogen sulfate, hydrochloride and besylate (crystalline) contents at 40 and 60° C. and 75% relative humidity over 15 days was tested. The results are shown in the attached FIG. 4.

One can see that the besylate salt (clopidogrel benzene sulfonate) has the best stability values both at 40 and 60° C.

### EXAMPLE 5

Adsorbate of (S)-(+)-Clopidogrel Besylate on Calcium Gluconate as Carrier Material

With vigorous stirring, a solution of 11 g (69.5 mmol) of anhydrous benzene sulfonic acid in 100 ml of cold anhydrous diethyl ether was slowly dropped into a solution of 19.7 g (61.4 mmol) of (S)-(+)-clopidogrel in 300 ml of anhydrous diethyl ether at 3° C. Then a premixed slurry of 28 g of calcium gluconate in cold anhydrous diethyl ether is added slowly. The crystal paste obtained after completion of the addition is removed by suction, washed with ice-cold anhydrous diethyl ether and then dried.

A white free-flowing powder is obtained.

### EXAMPLE 6

Adsorbate of (S)-(+)-Clopidogrel Besylate on Silica Gel/Mannitol as Carrier Material

20 g (62.3 mmol) of (S)-(+)-clopidogrel and 11 g (69.5 mmol) of anhydrous benzene sulfonic acid are reacted in 200 ml of anhydrous diethyl ether at a temperature of 2 to 3° C. Then a slurry of 2 g of silicic acid and 20 g of mannitol in 100 ml anhydrous diethyl ether is slowly added. The adsorbate thus obtained is removed by suction in cold conditions, washed with ice-cold anhydrous diethyl ether and then dried. 39 g of a white free-flowing powder are obtained.

### EXAMPLE 7

Adsorbate of (S)-(+)-Clopidogrel Mesylate on Silica Gel/Mannitol as Carrier Material

A solution of 5.85 g (60.8 mmol) anhydrous methane sulfonic acid in 100 ml of cold anhydrous diethyl ether is slowly (about 30 min.) dropped into a solution of 19.5 g (60.7 mmol) of (S)-(+)-clopidogrel in 300 ml of anhydrous diethyl ether at 3° C. Then a premixed slurry of 1.95 g of silicic acid and 19.5 g of mannitol in cold anhydrous diethyl ether is added slowly. The adsorbate obtained after completion of the addition is removed by suction, washed with ice-cold anhydrous diethyl ether and then dried.

30 of a free-flowing white powder are obtained.

### EXAMPLE 8

Adsorbate of (S)-(+) Clopidogrel Mesylate on Mannitol as Carrier Material

19.5 g (60.7 mmol) of (S)-(+)-clopidogrel and 5.85 g (60.8 mmol) of methane sulfonic acid are reacted in an analogous manner to example 7. Then a premixed slurry of 19.5 g of mannitol in cold anhydrous diethyl ether is added slowly. The adsorbate obtained after completion of the addition is removed by suction, washed with ice-cold anhydrous diethyl ether and then dried.

29.7 g of a free-flowing white powder are obtained.

### EXAMPLE 9

Adsorbate of (S)-(+) Clopidogrel on Silica Gel/Maize Starch as Carrier Material

A solution of 5 g (15.6 mmol) of (S)-(+)-clopidogrel in anhydrous dichloromethane is slowly dropped into a suspension of 2 g of Aerosil 200 in CH₂Cl₂. After one hour, a suspension of 4 g of gelatinised maize starch in anhydrous dichloromethane is added with stirring. After completion of the addition, the solvent is drawn off, resulting in a pure white solid which is then dried under vacuum for 12 hours.

A pure white, free-flowing powder having a 45.5% load of active ingredient is obtained.

Repetition of this experiment using 8 g of gelatinised maize starch resulted in a free-flowing powder having a 33.3% load of active ingredient.

### EXAMPLE 10

Two different methods were used to prepare adsorbates of the clopidogrel salts. In the first process, the salt is dissolved in a suitable solvent and the adsorbent suspended in this solution.

In a second series of experiments, the clopidogrel was dissolved in a suitable solvent, the adsorbent added and the salt precipitated onto the carrier material.

In all of these experiments, lactose (Lactopress®), mannitol (Mannogem®) and cellulose (Celphere®) were used as adsorbents.

The following experiments were conducted.

Clopidogrel-Salt Adsorbates with Isolation of the Salt

a) Clopidogrel Besylate Adsorbates

1.5 g (3.1 mmol) of clopidogrel besylate are dissolved in 20 ml of acetone and 1.5 g of adsorbent added. The solvent is drawn off, the residue briefly slurried with MTB ether and then dried under vacuum.

b) Clopidogrel Hydrochloride Adsorbates

500 mg (1.4 mmol) of clopidogrel hydrochloride are dissolved in 10 ml of acetone. 500 mg of adsorbent are added and stirred. The solvent is drawn off and the residue dried under vacuum.

c) Clopidogrel Hydrogen Sulfate Adsorbates

500 mg (1.2 mmol) of clopidogrel hydrogen sulfate are dissolved in 10 ml of acetone. 500 mg of adsorbent are added and stirred. The solvent is drawn off and the residue dried under vacuum.

Clopidogrel-Salt Adsorbates Without Prior Isolation of the Salts

1. Diethyl ether as the solvent

a) Clopidogrel Besylate Adsorbates

4.018 g (12.5 mmol) of clopidogrel base are dissolved in 20 ml of diethyl ether. 6 g of adsorbent and 1.977 g (12.5 mmol) of benzene sulfonic acid are added in 20 ml of ether. The solid product is removed by suction, washed with ether and dried under vacuum.

b) Clopidogrel Mesylate Adsorbates

4.018 g (12.5 mmol) of clopidogrel base are dissolved in 20 ml of diethyl ether. 6 g of adsorbent and 1.2 g (12.5 mmol) of methane sulfonic acid are added in 20 ml of ether. The solid product is removed by suction, washed with ether and dried under vacuum.

c) Clopidogrel Hydrochloride Adsorbates

3 g (9.3 mmol) of clopidogrel base are dissolved in 31 ml of diethyl ether. 3 g of adsorbent are added and hydrogen chloride gas introduced. The solid product is removed by suction, washed with ether and dried under vacuum.

2. Methyl-tert-butyl ether (MTB ether) as the solvent

a) Clopidogrel Besylate Adsorbates

4.018 g (12.5 mmol) of clopidogrel base are dissolved in 40 ml of MTB ether. 6 g of adsorbent and 1.977 g (12.5 mmol) of benzene sulfonic acid are added in 50 ml of MTB ether. The solid product is removed by suction, washed with MTB ether and dried under vacuum.

b) Clopidogrel Mesylate Adsorbates

4.018 g (12.5 mmol) of clopidogrel base are dissolved in 40 ml of MTB ether. 6 g of adsorbent and 1.2 g (12.5 mmol) of methane sulfonic acid are added in 50 ml of MTB ether. The solid product is removed by suction, washed with MTB ether and dried under vacuum.

### EXAMPLE 11

The stability of the adsorbates obtained according to example 10 was tested. The adsorbates kept their powder form at room temperature and did not change colour over more than two months.

The decrease of the active ingredient content during 15 days of storage at 40 and 60° C., respectively, and 75% of relative humidity was measured. The results are summarised in the following table 5 [content after 15 days (initial value normalised to 100%)].

TABLE 5

| | 40° C. | 60° C. |
|---|---|---|
| Clopidogrel hydrochloride | | |
| Pure salt | 93.66 | 42.54 |
| Lactopress | 99.78 | 54.89 |
| Celpher | 92.81 | 43.74 |
| Clopidogrel mesylate | | |
| Pure salt | 97.56 | 17.11 |
| Lactopress | 83.33 | 59.39 |
| Mannogem | 105.51 | 21.76 |
| Clopidogrel besylate | | |
| Pure salt | 103.32 | 66.48 |
| Lactopress/diethyl ether | 106.91 | 94.47 |
| Lactopress/MTB ether | 94.74 | 92.58 |

It is evident that the adsorbates have improved stability vis-à-vis the free salts, especially at elevated temperatures.

### EXAMPLE 12

Adsorbates prepared according to example 10 may be compressed directly into tablets. This is illustrated by the following sample formulations. The amounts of the other adjuvants used in the following examples are known to a skilled practitioner and may be taken from standard works on the formulation of tablets, such as Ritschel et al., “Die Tablette”, Editio Cantor—Aulendorf, 2^{nd} ed., 2002.

a) Clopidogrel Besylate Microcrystalline Cellulose Adsorbate

Clopidogrel tablets having a total weight of 275 mg and the following composition were prepared from the adsorbate by means of direct compression:

| | | |
|---|---|---|
| Clopidogrel besylate microcrystalline cellulose adsorbate | 219.54 | mg |
| (which corresponds to 75 mg of Clopidogrel base) | | |
| Adjuvants (lubricants, fillers, disintegration promoters, | ad 275 | mg |
| flow regulators, humectants) | | |

Characteristics of the compactible mixture and of the tablets:

| | |
|---|---|
| Compressibility and fluidity | satisfactory to good |
| Mean hardness | 101 N |
| Abrasion | 0.11% |
| Disintegration time | 65 sec. |
| Release | 100% after 30 min. |

The tablets thus obtained may also be provided with a coating such as an enteric coating or a coating to mask the taste.

b) Clopidogrel Besylate Mannitol Adsorbate

Clopidogrel tablets having a total weight of 275 mg and the following composition were prepared from the adsorbate by means of direct compression:

| | | |
|---|---|---|
| Clopidogrel besylate mannitol adsorbate | 219.54 | mg |
| (which corresponds to 75 mg of Clopidogrel base) | | |
| Adjuvants (lubricants, fillers, disintegration promoters, | ad 275 | mg |
| flow regulators, humectants) | | |

Characteristics of the compactible mixture and of the tablets:

| | |
|---|---|
| Compressibility and fluidity | satisfactory to good |
| Mean hardness | 106 N |
| Abrasion | 0.15% |
| Disintegration time | 62 sec. |
| Release | 100% after 30 min. |

The tablets thus obtained may be provided with a coating such as an enteric coating or a coating to mask the taste.

c) Clopidogrel Besylate Lactose Adsorbate

Clopidogrel tablets having a total weight of 275 mg and the following composition were prepared from the adsorbate by means of direct compression:

| | | |
|---|---|---|
| Clopidogrel besylate lactose adsorbate | 219.54 | mg |
| (which corresponds to 75 mg of Clopidogrel base) | | |
| Adjuvants (lubricants, fillers, disintegration promoters, | ad 275 | mg |
| flow regulators, humectants) | | |

Characteristics of the compactible mixture and of the tablets:

| | |
|---|---|
| Compressibility and fluidity | satisfactory to good |
| Mean hardness | N 96 |
| Abrasion | 0.21% |
| Disintegration time | sec. 76 |
| Release | 100% after 30 min. |

The tablets thus obtained may be provided with a coating such as an enteric coating or a coating to mask the taste.

d) Clopidogrel Mesylate Mannitol Adsorbate

Clopidogrel tablets having a total weight of 275 mg and the following composition were prepared from the adsorbate by means of direct compression:

| | | |
|---|---|---|
| Clopidogrel mesylate mannitol adsorbate | 194.79 | mg |
| (which corresponds to 75 mg of Clopidogrel base) | | |
| Adjuvants (lubricants, fillers, disintegration promoters, | ad 275 | mg |
| flow regulators, humectants) | | |

Characteristics of the compactible mixture and of the tablets:

| | |
|---|---|
| Compressibility and fluidity | satisfactory to good |
| Mean hardness | N 98 |
| Abrasion | 0.21% |
| Disintegration time | sec. 55 |
| Release | 100% after 30 min. |

The tablets thus obtained may be provided with a coating such as an enteric coating or a coating to mask the taste.

e) Clopidogrel Mesylate Lactose Adsorbate

Clopidogrel tablets having a total weight of 275 mg and the following composition were prepared from the adsorbate by means of direct compression:

| | | |
|---|---|---|
| Clopidogrel mesylate lactose adsorbate | 194.79 | mg |
| Adjuvants (lubricants, fillers, disintegration promoters, | ad 275 | mg |
| flow regulators, humectants) | | |

Characteristics of the compactible mixture and of the tablets:

| | |
|---|---|
| Compressibility and fluidity | satisfactory to good |
| Mean hardness | N 88 |
| Abrasion | 0.22% |
| Disintegration time | sec. 72 |
| Release | 100% after 30 min. |

The tablets thus obtained may be provided with a coating such as an enteric coating or a coating to mask the taste.

f) Clopidogrel HCl Lactose Adsorbate

Clopidogrel tablets having a total weight of 275 mg and the following composition were prepared from the adsorbate by means of direct compression:

| | | |
|---|---|---|
| Clopidogrel HCl lactose adsorbate | 167.0 | mg |
| Adjuvants (lubricants, fillers, disintegration promoters, | ad 275 | mg |
| flow regulators, humectants) | | |

Characteristics of the compactible mixture and of the tablets:

| | |
|---|---|
| Compressibility and fluidity | satisfactory to good |
| Mean hardness | N 95 |
| Abrasion | 0.20% |
| Disintegration time | sec. 75 |
| Release | 100% after 30 min. |

The tablets thus obtained may be provided with a coating such as an enteric coating or a coating to mask the taste.

g) Clopidogrel HCl Microcrystalline Cellulose Adsorbate

Clopidogrel tablets having a total weight of 275 mg and the following composition were prepared from the adsorbate by means of direct compression:

| | | |
|---|---|---|
| Clopidogrel HCl microcrystalline cellulose adsorbate | 167.0 | mg |
| (corresponds to 75 mg of clopidogrel base) | | |
| Adjuvants (lubricants, fillers, disintegration promoters, | ad 275 | mg |
| flow regulators, humectants) | | |

Characteristics of the compactible mixture and of the tablets:

| | |
|---|---|
| Compressibility and fluidity | satisfactory to good |
| Mean hardness | N 100 |
| Abrasion | 0.13% |
| Disintegration time | sec. 65 |
| Release | 100% after 30 min. |

The tablets thus obtained may also be provided with a coating such as an enteric coating or a coating to mask the taste.
h) Clopidogrel Hydrogen Sulfate Microcrystalline Cellulose Adsorbate

Clopidogrel tablets having a total weight of 275 mg and the following composition were prepared from the adsorbate by means of direct compression:

| | | |
|---|---|---|
| Clopidogrel hydrogen sulfate microcrystalline cellulose | 195.75 | mg |
| adsorbate (corresponds to 75 mg of clopidogrel base) | | |
| Adjuvants (lubricants, fillers, disintegration promoters, | ad 275 | mg |
| flow regulators, humectants) | | |

Characteristics of the compactible mixture and of the tablets:

| | |
|---|---|
| Compressibility and fluidity | satisfactory to good |
| Mean hardness | N 108 |
| Abrasion | 0.12% |
| Disintegration time | sec. 78 |
| Release | 98% after 30 min. |

The tablets thus obtained may also be provided with a coating such as an enteric coating or a coating to mask the taste.

i) Clopidogrel Hydrogen Sulfate Mannitol Adsorbate

Clopidogrel tablets having a total weight of 275 mg and the following composition were prepared from the adsorbate by means of direct compression:

| | | |
|---|---|---|
| Clopidogrel hydrogen sulfate mannitol adsorbate | 195.75 | mg |
| (corresponds to 75 mg of clopidogrel base) | | |
| Adjuvants (lubricants, fillers, disintegration promoters, | ad 275 | mg |
| flow regulators, humectants) | | |

Characteristics of the compactible mixture and of the tablets:

| | |
|---|---|
| Compressibility and fluidity | satisfactory to good |
| Mean hardness | N 110 |
| Abrasion | 0.13% |
| Disintegration time | sec. 80 |
| Release | 98% after 30 min. |

The tablets thus obtained may also be provided with a coating such as an enteric coating or a coating to mask the taste.

j) Clopidogrel Hydrogen Sulfate Lactose Adsorbate

Clopidogrel tablets having a total weight of 275 mg and the following composition were prepared from the adsorbate by means of direct compression:

| | | |
|---|---|---|
| Clopidogrel hydrogen sulfate lactose adsorbate | 195.75 | mg |
| (corresponds to 75 mg of clopidogrel base) | | |
| Adjuvants (lubricants, fillers, disintegration promoters, | ad 275 | mg |
| flow regulators, humectants) | | |

Characteristics of the compactible mixture and of the tablets:

| | |
|---|---|
| Compressibility and fluidity | satisfactory to good |
| Mean hardness | N 109 |
| Abrasion | 0.13% |
| Disintegration time | sec. 80 |
| Release | 98% after 30 min. |

The tablets thus obtained may also be provided with a coating such as an enteric coating or a coating to mask the taste.

1. The salt of a sulfonic acid with clopidogrel at least part of which is present in crystalline form.

2. The salt of a sulfonic acid with clopidogrel which is preparable by precipitating the salt from a clopidogrel solution, the solvent comprising a hydrocarbon and/or an ether.

3. The salt according to claim 2 wherein the solvent comprises toluene, dioxane, methyl-tert-butyl ether and/or diethyl ether.

4. The salt according to claim 2 at least part of which is present in crystalline form.

5. The salt according to claim 1 wherein the sulfonic acid is selected from the group consisting of methane sulfonic acid, ethane sulfonic acid, benzene sulfonic acid, toluene sulfonic acid and naphthalene sulfonic acid.

6. The salt according to claim 1 which contains solvent molecules.

7. The salt according to claim 5 wherein the solvent is selected from toluene and dioxane.

8. The salt according to claim 7 which is clopidogrel besylate, is present in crystalline form and contains toluene, the 10 most intensive peaks of the X-ray powder spectrum of this salt having the following 2θ values

| Relative intensity | 2θ |
|---|---|
| 99.11 | 10.78 |
| 100.00 | 12.08 |
| 96.77 | 16.09 |
| 62.57 | 16.66 |
| 84.58 | 20.22 |
| 93.53 | 21.50 |
| 66.00 | 22.56 |
| 78.33 | 22.91 |
| 81.82 | 23.45 |
| 56.15 | 24.92 |

9. The salt according to claim 8 which has the X-ray powder spectrum shown in FIG. 1.

10. The salt according to claim 7 which is clopidogrel besylate, is present in crystalline form and contains dioxane, the 10 most intensive peaks of the X-ray powder spectrum of this salt having the following 2θ values

| Relative intensity | 2θ |
|---|---|
| 51.66 | 10.78 |
| 54.15 | 10.87 |
| 90.13 | 12.13 |
| 50.83 | 14.34 |
| 50.27 | 16.43 |
| 76.03 | 21.57 |
| 81.19 | 22.87 |
| 100.00 | 23.06 |
| 54.18 | 23.72 |
| 54.05 | 25.17 |

11. The salt according to claim 10 which has the X-ray powder spectrum shown in FIG. 2.

12. The salt according to claim 5 which is clopidogrel tosylate, the 10 most intensive peaks of the X-ray powder spectrum of this salt having the following 2θ values

| Relative intensity | 2θ |
|---|---|
| 80.54 | 13.13 |
| 83.15 | 13.28 |
| 67.75 | 17.28 |
| 70.05 | 17.64 |
| 73.78 | 18.96 |
| 84.65 | 19.21 |
| 100.00 | 19.48 |
| 75.95 | 19.87 |
| 71.09 | 20.12 |
| 86.48 | 25.06 |

13. The salt according to claim 12 which has the X-ray powder spectrum shown in FIG. 3.

14. A method for preparing a salt according to claim 1 wherein the salt is precipitated from a solution of the clopidogrel and the solvent comprising a hydrocarbon and/or an ether.

15. A method according to claim 14 wherein the solvent comprises toluene, dioxane, methyl-tert-butyl ether and/or diethyl ether.

16. A method for purifying clopidogrel wherein contaminated clopidogrel or a salt thereof, optionally after release of the clopidogrel base, is converted into the salt of a sulfonic acid with clopidogrel and, if desired, the clopidogrel base is then released from the isolated salt of the sulfonic acid and/or converted into another salt.

17. The use of a salt according to claim 1 for preparing a pharmaceutical formulation.

18. A pharmaceutical formulation comprising a salt according to claim 1.

19. Active ingredient particles comprising a solid adsorbent and clopidogrel or a pharmaceutically acceptable salt thereof adsorbed thereon.

20. Active ingredient particles according to claim 19 wherein the salt is selected from the group consisting of hydrogen sulfate, hydrochloride, mesylate, besylate and tosylate and napsylate.

21. Active ingredient particles according to claim 19 wherein the adsorbent is Lactopress.

22. The use of active ingredient particles according to claim 19 for preparing a pharmaceutical formulation.

23. A pharmaceutical formulation comprising active ingredient particles according to claim 19.

24. A method for preparing active ingredient particles as defined in claim 19, comprising the recovery of the active ingredient particles from a solvent in which the adsorbent is insoluble or poorly soluble and the clopidogrel or the salt thereof is soluble.

25. A method according to claim 24 comprising suspending the adsorbent in the solvent, dissolving the clopidogrel or the salt thereof in the solvent and recovering the active ingredient particles.

26. A method according to claim 24 wherein the active ingredient particles are recovered by evaporation of the solvent.

27. A method according to claim 24 wherein the clopidogrel and an acid are mixed with the suspension of the adsorbent.

28. A method according to claim 24 wherein the last stage of the synthesis of clopidogrel or a pharmaceutically acceptable salt thereof is carried out in the presence of the adsorbent.
